(19) United States
(12) Patent Application Publication
(10) Pub. No.: US 2017/0252369 A1
(43) Pub. Date: Sep. 7, 2017

(54) METHOD FOR TREATING PARKINSON DISEASE, NEURODEGENERATIVE DISORDERS, AND AUTOIMMUNE DISEASES

(71) Applicant: Frederick J. Sawaya, Farmington Hills, MI (US)

(72) Inventor: Frederick J. Sawaya, Farmington Hills, MI (US)

(21) Appl. No.: 15/059,877

(22) Filed: Mar. 3, 2016

(51) Int. Cl. A61K 33/04

(52) U.S. Cl. CPC A61K 33/04

(57) ABSTRACT

The embodiments herein relate to a therapeutic method for the treatment of Parkinson disease, other neurodegerative disorders, and autoimmune diseases. The method includes providing sodium thiosulfate through any method of administration (orally, intravenously, through inhalation, intravesically, vaginally, rectal route, sublingually, ophthalmically, or topically) and is given with one or more pharmaceutically acceptable carriers or excipients. The sodium thiosulfate safely and endogenously generates H₂S in sufficient quantities in the human body to therapeutically treat damaged brain neurons.

### BACKGROUND

Technical Field of Invention

The present invention generally relates to a method for the treatment of Parkinson disease, neurodegenerative disorders and other autoimmune diseases.

Description of Related Art

Parkinson disease (PD), is a degenerative disorder of the central nervous system mainly affecting the motor system. The motor symptoms result from the death of dopamine-generating cells in the substantia nigra (SN). The causes of this cell death are largely unknown. Early in the course of the disease, the most obvious symptoms are movement-related; these include shaking, rigidity, slowness of movement and difficulty with walking and gait. Later, thinking and behavioral problems may arise, with dementia commonly occurring in the advanced stages of the disease. Depression is the most common psychiatric symptom. Other symptoms include sensory, sleep and emotional problems. Parkinson's disease is more common in older people, with most cases occurring after the age of 50; when it is seen in young adults, it is called Young Onset PD (YOPD).

The main motor symptoms are collectively called parkinsonism, or a “parkinsonian syndrome.” The disease can be either primary or secondary. Primary Parkinson's disease is referred to as idiopathic (having no known cause), although some atypical cases have a genetic origin, while secondary parkinsonism is due to known causes like toxins. Many risks and protective factors have been investigated: the clearest evidence is for an increased risk of PD in people exposed to certain pesticides and a reduced risk in tobacco smokers. The pathology of the disease is characterized by the accumulation of a protein into Lewy bodies in neurons, and insufficient formation and activity of dopamine in certain parts of the midbrain. Where the Lewy bodies are located is often related to the expression and degree of the symptoms of an individual. Diagnosis of typical cases is mainly based on symptoms, with tests such as neuroimaging being used for confirmation.

Treatments, typically the medications L-DOPA and dopamine agonists, improve the early symptoms of the disease. As the disease progresses and dopaminergic neurons continue to be lost, these drugs eventually become ineffective at treating the symptoms and at the same time produce a complication marked by involuntary writhing movements. Diet and some forms of rehabilitation have shown some effectiveness at improving symptoms. Surgery and deep brain stimulation have been used to reduce motor symptoms as a last resort in severe cases where drugs are ineffective. Research directions include investigations into new animal models of the disease and of the potential usefulness of gene therapy, stem cell transplants and neuroprotective agents. Medications to treat non-movement-related symptoms of PD, such as sleep disturbances and emotional problems, also exist.

Levodopa (L-DOPA) has been the most widely used treatment since 1967. L-DOPA is converted into dopamine in the dopaminergic neurons by the enzyme dopa decarboxylase. Since motor symptoms are produced by a lack of dopamine in the substantia nigra, the administration of L-DOPA temporarily diminishes the motor symptoms.

Recent research results have reported that hydrogen sulfide (H₂S) is effective at stimulating dopamine production in the substantia nigra (SN), in protecting SN neurons from further attack by activated glial cells, and in stimulating the repair and restoration of SN neurons. That same research notes that there is no known safe way to directly introduce exogenous H₂S into the human body, and laments that while the human body endogenously generates H₂S from cysteine, the maximum endogenous production rate is limited by a governing mechanism and never reaches therapeutic quantities.

Hence there is a need to develop a new compound or drug that can be safely introduced to the human body, and readily converts to H₂S within the human body in therapeutic quantities.

The above mentioned shortcomings, disadvantages and problems are addressed herein, as detailed below.

### SUMMARY OF THE INVENTION

The primary object of the embodiments herein is to provide a method for the treatment of Parkinson disease and other neurogenerative disorders and autoimmune diseases.

Another object of the embodiments herein is to provide a method that safely and endogenously generates H₂S in sufficient quantities to therapeutically treat Parkinson's-damaged substantia nigra neurons, leading to restoration of endogenous dopamine production.

Yet another object of the embodiments herein is to provide a method of treatment that safely provides therapeutic quantities of H₂S that effectively crosses the blood brain barrier to reach and treat damaged neurons.

The embodiments herein provide a method for the treatment of Parkinson's disease. The method comprises introducing a therapeutically effective amount of a sulfur compound via any route of administration and with any acceptable carrier or excipient. The sulfur compound is sodium thiosulfate (Na₂S₂O₃) (“STS”). The sulfur compound generates H₂S in a human body at sufficient quantities to therapeutically treat Parkinson's-damaged substantia nigra neurons.

### DETAILED DESCRIPTION OF THE EMBODIMENTS

The various embodiments herein provide a method for the treatment of Parkinson's disease neurodegenerative disease and autoimmune disease and, in particular, Parkinson disease. The method comprises introducing a therapeutically effective amount of a sulfur compound, the sulfur compound being sodium thiosulfate. The compound is not altered by any known excipient, emollient, or diluent used to effect intravenous, oral, topical, or other modes or routes of administration. The method does not rely upon any particular mode or excipient.

The present specification uses terms and abbreviations and molecular formulas such as “sodium thiosulfate” “STS” and Na₂S₂O₃, “hydrogen sulfide” and “H₂S”.

Orally administered STS is partially transformed into H₂S by digestive enzymes and stomach acids. The STS and H₂S are then partially absorbed by the intestine into the bloodstream.

STS that is administered by non-oral routes including intravenously, topically, intracutaneously, appear in the bloodstream unaltered by the route of administration or excipient used in the admistration. In the bloodstream, STS is partially transformed into H₂S through the action of thiosulfate reductase generated by the liver, kidneys, and testes, and is also produced within the mitochondria of the human cell. The enzyme is a membrane-bound enzyme. When STS comes in contact with enzyme thiosulfate reductase, a chemical reaction occurs and hydrogen sulfide is produced as a reduction product.

The humans affected by Parkinson disease have impaired substantia nigra (SN) neurons. The H₂S, when safely generated from STS in the human body, crosses the blood-brain barrier and therapeutically treats Parkinson's-damaged substantia nigra neurons.

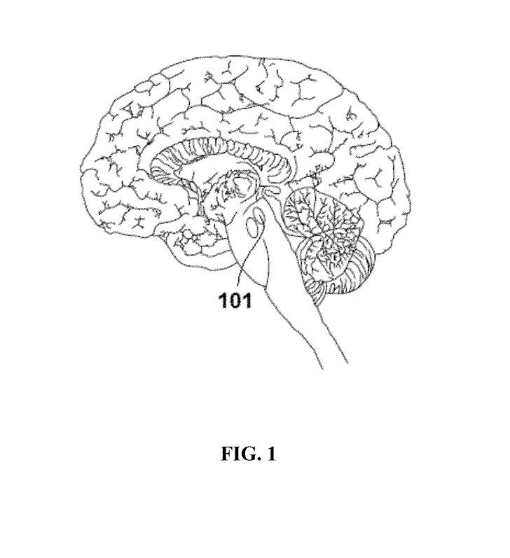

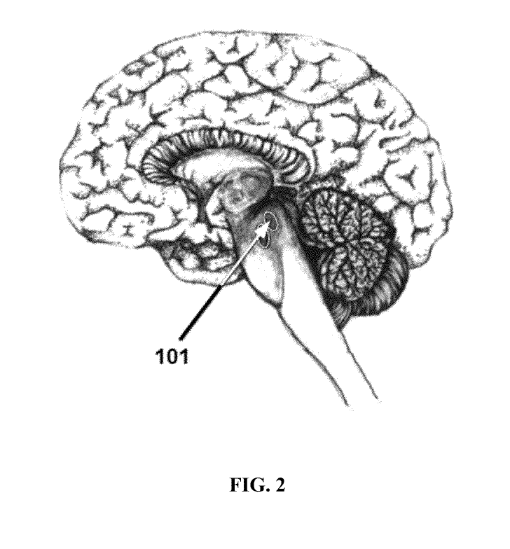

FIG. 1 and FIG. 2 shows a diagram of the human brain highlighting the position of the substantia niagra region. With respect to FIG. 1 and FIG. 2, the anatomical location of the substantia nigra neurons 101 can be seen. The composition of the present invention treats the substantia nigra neurons 101 with the active sulfur delivered by STS and H₂S.

Many changes and modifications may be made within the scope of the embodiments herein without departing from the spirit thereof, and the embodiments herein include all such modifications.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims.

1. A therapeutic method for the treatment of damaged substantia nigra in brain of a human body, comprising:
introducing a therapeutically effective amount of a sodium thiosulfate (i.e. Na₂S₂O₃ or STS) compound generating hydrogen sulfide (H₂S) into the human body through a method of administration with an acceptable pharmaceutical excipient restore endogenous dopamine production; and
wherein the method of administration is carried out one of orally or non-orally route to allow absorption of the sodium thiosulfate (i.e. Na₂S₂O₃ or STS) compound generating hydrogen sulfide (H₂S) in a bloodstream through a thiosulfate reductase generated by liver, kidneys, testes, intestine, or mitochondria in a cell of the human body to therapeutically treat damaged substantia nigra in the brain of the human body.

2. (canceled)

3. (canceled)

4. (canceled)

5. (canceled)

6. (canceled)
